(19) United States
(12) Patent Application Publication
(10) Pub. No.: US 2003/0054029 A1
(43) Pub. Date: Mar. 20, 2003

(54) IIB/IIIA ANTAGONISTS CO-ADMINISTERED WITH ASPIRIN

(76) Inventors: ROBERT J. ANDERS, VERNON HILLS, IL (US); LARRY P. FEIGEN, WAUCONDA, IL (US); MARK N, MILTON, GURNEE, IL (US); PETER F. SMITH, GURNEE, IL (US)

Correspondence Address: ROGER A WILLIAMS, G D SEARLE AND COMPANY, CORPORATE PATENT LAW DEPARTMENT, P O BOX 5100, CHIGACO, IL, 60680

(21) Appl. No.: 08/825,079

(22) Filed: Mar. 27, 1997

(51) Int. Cl. A61K 31/60; A61K 9/20

(52) U.S. Cl. 424/465; 514/165

(57) ABSTRACT

The present invention is directed to coadministration of the fibrinogen receptor antagonists 3S-[[4-[[4-aminoiminomethyl)phenyl]amino]-1,4-dioxobutyl]amino]-4-pentynoic acid or ethyl 3S-[[4-[[4-(aminoiminomethyl)phenyl]amino]-1,4-dioxobutyl]amino]-4-pentynoate together with an anti-platelet agent such as aspirin and/or an anti-coagulant such as heparin.

### FIELD OF THE INVENTION

This invention relates to the coadministration of a IIb/IIIa receptor antagonist with aspirin for use in inhibiting platelet aggregation when administered to mammals which coadministration significantly lowers the required dosage of IIb/IIIa antagonist to be administered for effectively inhibiting platelet aggregation.

### BACKGROUND OF THE INVENTION

Fibrinogen is a glycoprotein present as a normal component of blood plasma. It participates in platelet aggregation and fibrin formation in the blood clotting mechanism.

Platelets are cellular elements found in whole blood which also participate in blood coagulation. Fibrinogen binding to platelets is important to normal platelet function in the blood coagulation mechanism. When a blood vessel receives an injury, the platelets binding to fibrinogen will initiate aggregation and form a thrombus. Interaction of fibrinogen with platelets occurs through a membrane glycoprotein complex, known as GPIIb/IIIa; this is an important feature of the platelet function. Inhibitors of this interaction are useful in modulating or preventing platelet thrombus formation.

It is also known that another large glycoprotein named fibronectin, which is a major extracellular matrix protein, interacts with fibrinogen and fibrin, and with other structural molecules such as actin, collagen and proteoglycans. Various relatively large polypeptide fragments in the cell-binding domain of fibronectin have been found to have cell-attachment activity.

The activation of platelets and the resultant aggregation have been shown to be important factors in the pathogenesis of unstable angina pectoris, transient myocardial ischemia, acute myocardial infarction and atherosclerosis. In most of these serious cardiovascular disorders, intracoronary thrombus is present. The thrombus is generally formed by activated platelets that adhere and aggregate at the site of endothelial injury. Because of the relative contribution of activated platelets to aggregation and subsequent formation of an occlusive thrombus, antiplatelet agents have been developed that inhibit platelet aggregation but these previous agents have limited mechanisms of action. Current antiplatelet agents include aspirin (ASA), which mainly interrupts the thromboxane pathway; ticlopidine, which predominantly interferes with the ability of adenosine diphosphate (ADP) to stimulate platelets; and thromboxane A₂ synthetase inhibitors, which act against thromboxane A₂.

Attempts to inhibit platelet aggregation have resulted in the development of new agents that block fibrinogen (fgn) binding [at the arginine-glycine-aspartate (RGD) recognition sequence] to the glycoprotein (GP) IIb/IIIa receptor on activated platelets. The binding of fgn to the GPIIb/IIIa receptors is considered the final common pathway of platelet aggregation that leads to thrombus formation. A drug therapy that inhibits platelet aggregation induced by a variety of physiological agonists would provide even greater protection than that provided by the various agents listed above.

Fibrinogen receptor antagonists block the fibrinogen/platelet interaction at the glycoprotein (GP) IIb/IIIa receptors, inhibiting an essential step in thrombus formation. 3S-[[4-[[4-aminoiminomethyl)-phenyl]amino]-1,4-dioxobutyl]amino]-4-pentynoic acid, a fibrinogen receptor antagonist, is the active metabolite of ethyl 3S-[[4-[[4-(aminoiminomethyl)phenyl]amino]-1,4-dioxobutyl]amino]-4-pentynoate, an orally active antithrombotic agent now in clinical trials.

3S-[[4-[[4-aminoiminomethyl)phenyl]amino]-1,4-dioxobutyl]amino]-4-pentynoic acid is a GPIIb/IIIa receptor antagonist that blocks the binding of fibrinogen to the platelet and prevents platelet aggregation. Intravenous 3S-[[4-[[4-aminoiminomethyl)phenyl]amino]-1,4-dioxobutyl]amino]-4-pentynoic acid, aspirin (ASA), heparin and combinations of these agents were evaluated in an anesthetized canine model of thrombosis and inhibition of collagen-induced ex vivo platelet aggregation was determined. 3S-[[4-[[4-aminoiminomethyl)phenyl]-amino]-1,4-dioxobutyl]amino]-4-pentynoic acid prevents thrombotic occlusion and inhibits platelet aggregation in a dose-dependent manner. Surprisingly it has now been found antithrombotic effects are enhanced with treatment regimens of a reduced dose of 3S-[[4-[[4-aminoiminomethyl)phenyl]amino]-1,4-dioxobutyl]amino]-4-pentynoic acid combined with ASA and/or heparin whereas ASA, heparin and saline when used alone were ineffective in this model.

### SUMMARY OF THE INVENTION

The present invention is directed to coadministration of the fibrinogen receptor antagonists 3S-[[4-[[4-aminoiminomethyl)phenyl]amino]-1,4-dioxobutyl]amino]-4-pentynoic acid or ethyl 3S-[[4-[[4-(aminoiminomethyl)phenyl]amino]-1,4-dioxobutyl]amino]-4-pentynoate together with an anti-platelet agent such as aspirin and/or an anti-coagulant such as heparin. Such coadministration comprises administering a therapeutically effective amount of aspirin or heparin to a mammal in need of a platelet aggregation inhibitor which therapeutically effective amount significantly lowers the amount of 3S-[[4-[[4-aminoiminomethyl)phenyl]amino]-1,4-dioxobutyl]amino]-4-pentynoic acid or ethyl 3S-[[4-[[4-(aminoiminomethyl)phenyl]amino]-1,4-dioxobutyl]amino]-4-pentynoate needed to effectively inhibit platelet aggregation.

### DETAILED DESCRIPTION OF THE INVENTION

3S-[[4-[[4-aminoiminomethyl)phenyl]amino]-1,4-dioxobutyl]amino]-4-pentynoic acid of the formula

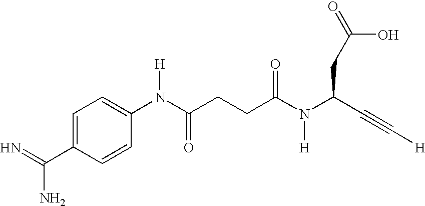

is the active metabolite of the ester ethyl 3S-[[4-[[4-(aminoiminomethyl)phenyl]amino]-1,4-dioxobutyl]amino]-4-pentynoate of the formula

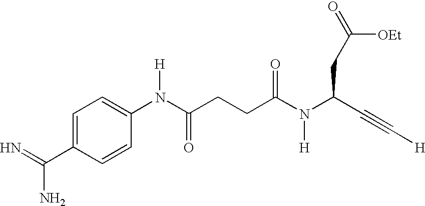

These compounds and their pharmaceutically acceptable salts are disclosed in U.S. Pat. No. 5,344,957 as platelet aggregation inhibitors which act by inhibition of glycoprotein IIb/IIIa.

These compounds are useful in inhibiting the binding of fibrinogen to blood platelets, inhibiting aggregation of blood platelets, treatment of thrombus formation or embolus formation, and in the prevention of thrombus formation or embolus formation. These compounds are useful as pharmaceutical agents for mammals, especially for humans. These compounds can be administered to patients where prevention of thrombosis by inhibiting binding of fibrinogen to the platelet membrane glycoprotein complex IIb/IIIa receptor is desired. These compounds can also be used to prevent or modulate the progress of myocardial infarction, unstable angina and thrombotic stroke, when longer-term treatment may be desirable. In addition, they may be useful in surgery on peripheral arteries (arterial grafts, carotid endarterectomy) and in cardiovascular surgery where manipulation of arteries and organs, and/or the interaction of platelets with artificial surfaces, leads to platelet aggregation and consumption. The aggregated platelets may form thrombi and thromboemboli. These compounds may be administered to surgical patients to prevent the formation of thrombi and thromboemboli.

Other applications of these compounds include prevention of platelet thrombosis, thromboembolism, reocclusion, and restenosis during and after thrombolytic therapy and prevention of platelet thrombosis, thromboembolism, reocclusion and restenosis after angioplasty of coronary and other arteries and after coronary artery bypass procedures.

These compounds are prepared according to the methodology disclosed in U.S. Pat. No. 5,344,957 and more specifically are prepared as follows.

### EXAMPLE 1

Ethyl 3S-[[4-[[4-(aminoiminomethyl)phenyl]-amino]-1,4-dioxobutyl]amino]-4-pentynoate

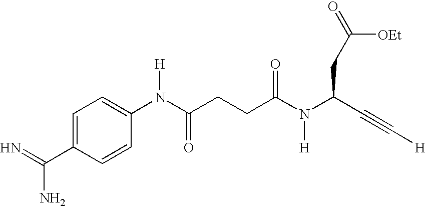

Step A

Preparation of 4-[[4-(aminoiminomethyl)phenyl]-amino]-4-oxobutanoic acid.

4-Aminobenzamidine di-HCl (25 g, 120 mmol), which is commercially available from Aldrich, was added to dry DMF (100 ml). To this solution dry pyridine (100 ml) and succinic anhydride (12 g, 120 mmol) followed by dimethylaminopyridine (DMAP 1.5 g, 0.012 mmol) were added. The product precipitated after heating for ½ hour at 100° C. The product was filtered, washed with water, acetonitrile and ether. The light solid was suspended in dioxane, 4N HCl in dioxane (100 ml) was added and the suspension was stirred for 1 hour, filtered and dried in a desiccator to give 28 g, (88%) of 4-[[4-(aminoiminomethyl)phenyl]-amino]-4-oxobutanoic acid as a white yellow solid which decomposes between 270° and 290° C.

Step B

Preparation of D,L-3-[[4-[[4-(aminoiminomethyl)phenyl]amino]-1,4-dioxobutyl]amino]-3-phenylpropionic acid.

4-([4-(Aminoiminomethyl)phenyl]-amino)-4-oxobutanoic acid hydrochloride prepared in Step A (1 g, 3.7 mmol) was added to dry DMF (35 ml) followed by N-methylmorpholine (0.39 g, 1 eq.) and isobutyl chloroformate (0.53 g, 3.9 mmol) at 25° C. The mixture was stirred for 5 minutes. (S)-ethyl 3-amino-4-pentynoate was added followed by diisopropylethylamine (0.68 mL; 3.9 mmol) and a catalytic amount of dimethylaminopyridine. After 1 hour, the solvent was removed under reduced pressure and the product was purified by reverse phase chromatography (0.05% TFA water/acetonitrile) to give the desired product ¹³C NMR (CD₃OD) δ 13.6, 30.3, 31.9, 38.1, 40.4, 61.0, 71.9, 82.0, 119.6, 122.5, 129.1, 144.8, 166.5, 170.3, 172.1, 172.2. The ratio of enantiomers was determined to be 98.2 by chiral HPLC using an AGP column.

Analysis Calc'd. for C₂₀H₂₆N₄O₄ plus 0.2 CF₃CO₂H, 0.8 HCl and 1.0H₂O: C, 51.59; H, 5.88; N, 13.08. Found: C, 51.68; H, 5.45; N, 12.89.

### EXAMPLE 2

3S-[[4-[[4-aminoiminomethyl)phenyl]amino]-1,4-dioxobutyl]amino]-4-pentynoic Acid

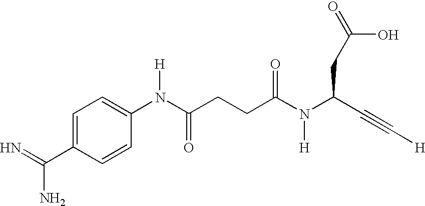

The title compound was prepared by treating the final product of the previous example with porcine liver esterase. Porcine liver esterase (200 μL, signal 11 mg/mL in 3.2 M (NH₄)₂SO₄ at pH=8) was added to the compound of Example 1 in 20 mL of 0.1 M phosphate buffer (pH=7.4). After 24 hours at 23° C., the reaction mixture was concentrated in vacuo. The residue was dissolved in 1N HCl (3 mL) and subsequently diluted with aceonitrile (5 ml). The product was purified by reverse phase HPLC using the conditions of Example 1 to afford the title compound. C NMR (CD₃OD) δ 29.9, 31.4, 37.7, 39.5, 71.1, 81.5, 119.2, 122.1, 128.3, 144.2, 166.2, 171.8, 172.0, 172.1. Optical Rotation [α]_{D} −33.7 (c 1.45, CH₃OH).

Analysis Calc'd. for C₁₆H₁₈N₄O₄ plus 1.85 HCl and 0.95H₂O: C, 46.32; H, 5.28; N, 13.50. Found: C, 46.51; H, 5.38; N, 13.52.

The following examples describe specific formulations used for tabletting the IIb/IIIa antagonists useful in the present invention.

### EXAMPLE 3

| Formulation | 2.5 mg | | | 5.0 mg | | |
|---|---|---|---|---|---|---|
| Compound of Example 1 | 2.75 | mg | (1.375%) | 5.5 | mg | (2.75%) |
| Avicel PH-302 | 186.25 | mg | | 183.5 | mg | |
| Starch 1500 | 8.0 | mg | (4%) | 8.0 | mg | (4%) |
| Talc | 2.0 | mg | (1%) | 2.0 | mg | (1%) |
| Mg Stearate | 1.0 | mg | (0.5%) | 1.0 | mg | 0.5% |
| Weight/Tablet | 200 | mg | | 200 | mg | |
| Opadry Coating | 5 | mg | | 5 | mg | |

### EXAMPLE 4

| Formulation | 10 mg | | | 25 mg | | |
|---|---|---|---|---|---|---|
| Compound of Example 1 | 11.0 | mg | (5.5%) | 27.5 | mg | (13.75%) |
| Avicel PH-302 | 178.0 | mg | | 161.5 | mg | |
| Starch 1500 | 8.0 | mg | (4%) | 8.0 | mg | (4%) |
| Talc | 2.0 | mg | (1%) | 2.0 | mg | (1%) |
| Mg Stearate | 1.0 | mg | (0.5%) | 1.0 | mg | 0.5% |
| Weight/Tablet | 200 | mg | | 200 | mg | |
| Opadry Coating | 5 | mg | | 5 | mg | |

The ingredients are milled, weighed, blended, tabletted and coated using conventional and well known tabletting methodology.

Total daily dose administered to a host in single or divided doses may be in amounts, for example, from 0.001 to 100 mg/kg body weight daily and more usually 0.01 to 10 mg/kg. Dosage unit compositions may contain such amounts of submultiples thereof to make up the daily dose.

The amount of active ingredient that may be combined with the carrier materials to produce a single dosage form will vary depending upon the host treated and the particular mode of administration.

It will be understood, however, that the specific dose level for any particular patient will depend upon a variety of factors including the activity of the specific compound employed, the age, body weight, general health, sex, diets, time of administration, route of administration, rate of excretion, drug combination, and the severity of the particular disease undergoing therapy.

The compounds useful in the present invention may be administered orally, parenterally, by inhalation spray, rectally, transdermally or topically in dosage unit formulations containing conventional nontoxic pharmaceutically acceptable carriers, adjuvants, and vehicles as desired.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols which are solid at ordinary temperature but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

In the present invention it has now been found that 3S-[[4-[[4-aminoiminomethyl)phenyl]amino]-1,4-dioxobutyl]amino]-4-pentynoic acid or ethyl 3S-[[4-[[4-(aminoiminomethyl)phenyl]amino]-1,4-dioxobutyl]amino]-4-pentynoate or pharmaceutically acceptable salts thereof can be co-administered with suitable anti-coagulants such as heparin or warfarin and/or anti-platelet agents, such as indomethacin, ibuprofen, naproxen, diclofenac, ticlopidine or aspirin while significantly reducing the dosage amount of 3S-[[4-[[4-aminoiminomethyl)phenyl]amino]-1,4-dioxobutyl]amino]-4-pentynoic acid or ethyl 3S-[[4-[[4-(aminoiminomethyl)phenyl]amino]-1,4-dioxobutyl]amino]-4-pentynoate needed to effectively inhibit platelet aggregation in a mammal in need of such treatment.

The term anti-coagulant agents, as used herein, denotes agents that inhibit blood coagulation. Such agents include warfarin or heparin, including low molecular weight heparin (LMWH), and pharmaceutically acceptable salts or prodrugs thereof. The heparin employed herein may be, for example, the sodium or sulfate salts thereof.

The term anti-platelet agents, as used herein, denotes agents that inhibit platelet function such as by inhibiting the aggregation, adhesion or granular secretion of platelets. Such agents include the various known non-steroidal anti-inflammatory drugs such as indomethacin, ibuprofen, naproxen, diclofenac, aspirin and piroxicam. Another suitable anti-platelet agent is ticlopidine. Aspirin (acetylsalicyclic acid or ASA), which has been well researched and widely used with good results, is the preferred agent.

Thromboembolic disorders are known to have a diverse pathophysiological makeup. Therefore, there is a need for a therapeutic approach to the treatment of these disorders which takes into account the diverse pathophysiological makeup of such diseases, and which includes components ameliorating each of the various pathophysiological aspects. A combination therapy containing an anti-coagulant agent such as heparin, or an antiplatelet agent such as aspirin, in combination with a IIb/IIIa antagonist such as 3S-[[4-[[4-aminoiminomethyl)phenyl]amino]-1,4-dioxobutyl]amino]-4-pentynoic acid or ethyl 3S-[[4-[[4-(aminoiminomethyl)phenyl]amino]-1,4-dioxobutyl]amino]-4-pentynoate, or pharmaceutically acceptable salts thereof can provide such a therapy.

In addition, by administering lower doses of each, which is feasible where an additive or synergistic effect is involved, the incidence of any side effects associated with each alone at higher doses may be significantly reduced.

In a preferred embodiment, the glycoprotein IIb/IIIa compounds used in this invention and the anti-coagulant agent and/or anti-platelet agent, can be administered at the same time (that is, together), or in any order, for example the IIb/IIIa antagonists used in this invention are administered first, followed by administration of the anti-coagulant agent and/or anti-platelet agent. When not administered at the same time, preferably the administration of the IIb/IIIa antagonists used in this invention and any anti-coagulant agent and/or anti-platelet agent occurs less than about one hour apart, more preferably less than about 30 minutes apart, even more preferably less than about 15 minutes apart, and most preferably less than about 5 minutes apart. Preferably, when an oral dosage form of each agent is available, administration of the combination therapy of the invention is oral. The terms oral agent, oral inhibitor, oral compound, or the like, as used herein, denote compounds which may be orally administered. Although it is preferable that the IIb/IIIa antagonist compounds of this invention and the anti-coagulant agent and/or anti-platelet agent, are both administered in the same fashion (that is, for example, both orally), if desired, they may each be administered in different fashions (that is, for example, one component of the combination product may be administered orally, and another component may be administered intravenously). The dosage of the combination products of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired, as described above.

Although the proper dosage of the agent of the combination therapy of this invention can be readily ascertainable by one skilled in the art, once possessed of the present disclosure, by way of general guidance, where the IIb/IIIa antagonist compounds useful in this invention are combined with anti-coagulant agents, for example, typically a daily dosage may be about 5 milligrams to 60 milligrams of the IIb/IIIa antagonist compounds useful in this invention and about 160 to 1500 milligrams of the anticoagulant, preferably about 5 to 40 milligrams of the IIb/IIIa antagonist compound useful in this invention and about 160 to 1000 milligrams of the anti-coagulants per day.

Where the IIb/IIIa antagonist compounds useful in the present invention are combined with another antiplatelet agent, by way of general guidance, typically a daily dosage may be about 5 to 60 milligrams of the IIb/IIIa antagonist compounds useful in the present invention and about 75 to 325 milligrams of the antiplatelet agent, preferably about 5 to 40 milligrams of the IIb/IIIa antagonist compounds useful in the present invention and about 160 to 325 milligrams of antiplatelet agents, per day.

While the normal dosage of IIb/IIIa antagonists would be in the range of about 20 mg to about 25 mg twice a day or higher, which may overlap the current combination therapy dose, it has now been found that the therapeutically effective amount of IIb/IIIa antagonist administered in the combination therapy, is less than the amount administered when the IIb/IIIa antagonist is administered alone, to achieve the same therapeutic effect.

As discussed above, where two or more of the foregoing therapeutic agents are co-administered with the IIb/IIIa antagonist compounds of this invention, generally the amount of each component in a typical daily dosage and typical dosage form may be reduced relative to the usual dosage of the agent when administered alone, in view of the additive or synergistic effect which would be obtained as a result of addition of further agents in accordance with the present invention.

Most preferably the IIb/IIIa antagonist compounds useful in the present invention are administered to humans in dosages of 5 mg, 10 mg, 15 mg and 20 mg twice a day and aspirin is given in dosages ranging from 160-325 mg once a day.

### In-Vitro Platelet Aggregation in PRP

Healthy male or female dogs were fasted for 8 hours prior to drawing blood; then 30 ml whole blood was collected using a butterfly needle and 30 cc plastic syringe with 3 ml of 0.129 M buffered sodium citrate (3.8%). The syringe was rotated carefully as blood was drawn to mix the citrate. Platelet-rich plasma (PRP) was prepared by centrifugation at 975×g for 3.17 minutes at room temperature allowing the centrifuge to coast to a stop without braking. The PRP was removed from the blood with a plastic pipette and placed in a plastic capped, 50 mL Corning conical sterile centrifuge tube which was held at room temperature. Platelet poor plasma (PPP) was prepared by centrifuging the remaining blood at 2000×g for 15 minutes at room temperature allowing the centrifuge to coast to a stop without braking. The PRP was adjusted with PPP to a count of 2-3×10⁸ platelets per mL. 400 uL of the PRP preparation and 50 uL of the compounds solution to be tested or saline were preincubated for 1 minute at 37° C. in an aggregometer (BioData, Horsham, Pa.). 50 uL of adenosine 5′ diphosphate (ADP) (50 um final concentration) was added to the cuvettes and the aggregation was monitored for 1 minute. All compounds are tested in duplicate. Results are calculated as follows: Percent of control=[(maximal OD minus initial OD of compound) divided by (maximal OD minus initial OD of control saline)]×100. The % inhibition=100−(percent of control).

The compounds tested and their median inhibitory concentrations (IC₅₀) are recorded in Table I. IC₅₀'s (dosage at which 50% of platelet aggregation is inhibited) were calculated by linear regression of the dose response curve. The assay results for the compounds of Examples 1 and 2 are set forth in Table 1, below.

TABLE 1

| | Dog PRP | Ex Vivo Effect |
|---|---|---|
| Example | IC₅₀ μm | after IG Admins. |
| 1 | 4.6 | + |
| 2 | 0.07 | + |

### Methods

Reagents

Lysine-ASA was obtained from Synthélabo (Brussels, Belgium). Collagen from equine tendon was purchased from Chrono-log Corporation (Havertown, Pa.). Sodium heparin from beef lung was obtained from Upjohn Company (Kalamazoo, Mich.). Saline was purchased from Baxter Health Corporation (Deerfield, Ill.). 3S-[[4-[[4-aminoiminomethyl)phenyl]amino]-1,4-dioxobutyl]amino]-4-pentynoic acid was synthesized at G. D. Searle & Co. (Skokie, Ill.).

Surgical Preparation and Instrumentation

Sixty-six mongrel dogs of either sex weighing between 14 and 26 kg were anesthetized by intravenous administration of pentobarbital sodium solution (30 mg/kg). A supplemental dose of the anesthetic (65 to 130 mg) was administered as required. The dogs were endotracheally intubated and placed on a respirator (Biological Research Apparatus, Comerio-Varese, Italy) with the stroke volume adjusted to 20 ml/kg and a frequency of 12 breaths/minute. Peripheral arterial blood pressure was monitored with a pressure transducer (Micron Instruments, Simi Valley, Calif.) connected to a catheter placed in the right femoral artery. A catheter was inserted into the right femoral vein for withdrawing blood samples and another was inserted into the left jugular vein for administering intravenous fluids. A left thoracotomy was performed in the fifth intercostal space and the heart was suspended in a pericardial cradle. A 2 to 3-cm segment of the left circumflex coronary artery (LCCA) was isolated distal to the first diagonal branch. The small intervening coronary branches over the isolated segment were ligated. The artery was instrumented from proximal to distal with an ultrasonic flow probe, a stimulation electrode, and a Goldblatt clamp. The flow probe was connected to a Doppler flow meter (Crystal Biotech, Hopkinton, Mass.) in order to monitor the mean and the phasic LCCA blood flow velocities. The stimulation electrode and its placement in the LCCA and the methodology to induce an occlusive coronary thrombus are described in detail in Mickelson et al. Circulation, 1990; 81:617-627; Shebuski et al., Circulation, 1990; 82:169-177; and Tschopp et al., Coronary Artery Des., 1993; 4:809-817. Briefly, the needle tip of the electrode was inserted into the LCCA, ensuring its contact with the intraluminal surface of the vessel just under the Goldblatt clamp. The clamp was adjusted to reduce the peak reactive hyperemia following a 10-second period of total occlusion, without affecting the baseline mean LCCA blood flow velocity. Continuous recordings of blood pressure and LCCA blood flow velocity (mean and phasic) were obtained on a multichannel recorder (Gould Inc., Cleveland, Ohio).

Experimental Protocol

Approximately 30 minutes after the preparation of the dogs, the study was continued by the administration of one of the treatments presented in Table 2.

TABLE 2
Treatment Regiments Administered to Anesthetized Dogs

| Group | Intravenous treatment |
|---|---|
| 1 | 1 × SCa (0.87//0.39 μg/kg/min) |
| 2 | 0.6 × SCa (0.52//0.23 μg/kg/min) |
| 3 | 0.5 × SCa (0.425//0.20 μg/kg/min) |
| 4 | ASA (2.8 mg/kg bolus) |
| 5 | Heparin^{a} |
| 6 | ASA + Heparin |
| 7 | 0.5 × SCa + ASA |
| 8 | 0.5 × SCa + Heparin |
| 9 | 0.5 × SCa + ASA + Heparin |
| 10 | 0.4 × SCa (0.34//0.16 μg/kg/min) + ASA + |
| | Heparin |
| 11 | Saline (0.9%, 0.1 ml/kg bolus) |

ASA = lysine-aspirin (adjustment was made for the lysine content); SCa = 3S-[[4-[[4-aminoiminomethyl) phenyl]-amino]-1,4-dioxobutyl]amino]-4-pentynoic acid (administered as a loading dose for 15 minutes, // then as a maintenance infusion).
^{a}= 200 units/kg (bolus) followed by 1000 units/hour (bolus). n = 6/group.

The 0.6×SCa, 0.5×SCa and 0.4×SCa doses in Table 2 represent the reduced doses of the highest dose of SCa tested. Each dog was utilized only once. At 30 minutes, the stimulation electrode was then connected in series with a 12 K Ohms-112 K Ohms variable resistor to the positive terminal of a 9-V battery. The electrical circuit was completed by securing a needle electrode into a subcutaneous site and to the negative terminal of the battery. The anodal current delivered to the tip of the stimulation electrode was monitored and maintained at 250 μA. The number and the frequency of cyclic flow variations (CFV) that preceded the formation of an occlusive thrombus were recorded. CFV were observed as spontaneous shifts in mean and phasic LCCA blood flow velocity, with the sudden return of these variables to baseline. Proper positioning of the electrode in the LCCA was confirmed by visual inspection at the end of the experiment. Each experiment lasted for 180 minutes of anodal current unless the dog died after an occlusive thrombus was formed. Lack of antithrombotic efficacy was established if zero flow in the LCCA was observed for a minimum of 30 minutes.

Ex vivo Platelet Aggregation and Platelet Counts

Peripheral venous blood was collected into citrated Vacutainer tubes (containing 0.3 ml of 3.8% sodium citrate solution) and platelet-rich plasma (PRP) was obtained by centrifugation (model Technospin R, Sorvall Instruments, DuPont, Wilmington, Del.) the blood at 266×g for 6 minutes at 24° C. Platelet-poor plasma (PPP) was obtained by further centrifugation at 2000×g for 10 minutes at 24° C. Samples were assayed on an aggregometer (model PAP-4, Bio/Data Corporation, Hatboro, Pa.) with PPP as the blank. The aggregations were performed by adding 50 μl of collagen (33.3 μl/ml final concentration) to 450 μl of PRP and measuring aggregation for 3 minutes. Blood samples used in platelet aggregation were collected at the following time periods: before treatment administration (baseline), immediately before anodal stimulation (at 30 minutes), at 60 minutes, then at 1-hour intervals to the end of experimentation. The blood samples at 60, 120 and 180 minutes were averaged (since the three blood samples yielded similar data) to obtain the steady-state platelet inhibition value for all comparisons except for saline and heparin (since no 120-minute sample was taken, 60 and 180 minute samples were used). Results are expressed as percent inhibition and represent steady-state conditions.

Venous blood for whole blood platelet counts was collected into Vacutainer tubes (containing 0.04 ml of 7.5 EDTA solution) at baseline. Platelet counts were determined with a Coulter counter (model S-Plus IV, Hialeah, Fla.).

Bioassay for SCa Plasma Levels

SCa plasma levels were determined from the blood samples used for platelet aggregation. Plasma levels of SCa were measured using a modification of a bioassay method previously described in Salyers et al., Throm. Res., 1994, 75:409-417. The bioassay used plasma from treated dogs as the source of inhibitor to be tested in vitro against normal (naive) platelets from donor dogs. Briefly, PRP from non-treated dogs was added to wells containing plasma samples from treated dogs in a 96 well microtiter plate. ADP (20 μM) was added to the platelet suspension in each well to induce aggregation. Optical density at 405 nanometers was measured on all wells simultaneously in a platereader (Thermomax microplate reader, Molecular Devices, Menlo Park, Calif.). The results were quantified by comparison to a standard inhibition curve prepared in plasma using known amounts of SCa.

Data Analysis

Data are expressed as mean±SEM. All tests for statistical significance were nonparametric. When dose-dependency was expected, that is, higher doses resulting in longer times to zero flow and greater percent inhibitions than lower doses, the data were analyzed by one-tailed chi-bar square trend tests. Other comparisons were made using either one- or two-tailed Dunnett tests. Differences were considered significant at p<0.05.

The three doses of 3S-[[4-[[4-aminoiminomethyl)phenyl]amino]-1,4-dioxobutyl]amino]-4-pentynoic acid were more efficacious in preventing an occlusive thrombus than ASA, heparin, ASA combined with heparin, or the saline control. Thrombosis did not occur in any of the dogs treated with 3S-[[4-[[4-aminoiminomethyl)phenyl]amino]-1,4-dioxobutyl]amino]-4-pentynoic acid (1×SCa). It did occur before the completion of the 180 minutes of current in all of the dogs treated with saline or ASA. The time to zero flow was significantly prolonged by the three doses of SCa (1×SCa, 180±0; 0.6×SCa, 158±15; 0.5×SCa, 130±22 minutes) relative to the treatment with ASA (64±7 minutes) and saline (58±7 minutes). The time to zero flow for 1×SCa was represented by 180±0 minutes; this time merely established the end of the experimental protocol with no occlusion. The time to zero flow was increased by heparin (114±16 minutes) and the combination of ASA with heparin (130±11 minutes) compared to the saline treatment but 1×SCa provided a significantly longer time to zero flow than either of these treatments.

A dose-dependent increase in the steady-state inhibition of platelet aggregation was obtained after the administration of the three dose regiments of SCa (1×SCa, 92±5; 0.6×SCa, 83±3; 0.5×SCa, 70±4%, respectively). The dose regimens of SCa leading to >90% inhibition of platelet aggregation either increased the time to zero flow or prevented thrombotic occlusion. Each regimen of SCa produced a level of inhibition that was significantly greater than that obtained from the heparin (13±2%), ASA combined with heparin (24±10%), or saline (9±2%) treatments. Only 1×SCa and 0.6×SCa significantly inhibited platelet aggregation relative to ASA (25±15%).

Effects of Decreased Doses of SCa Given in Combination with ASA, Heparin, or ASA Combined with Heparin

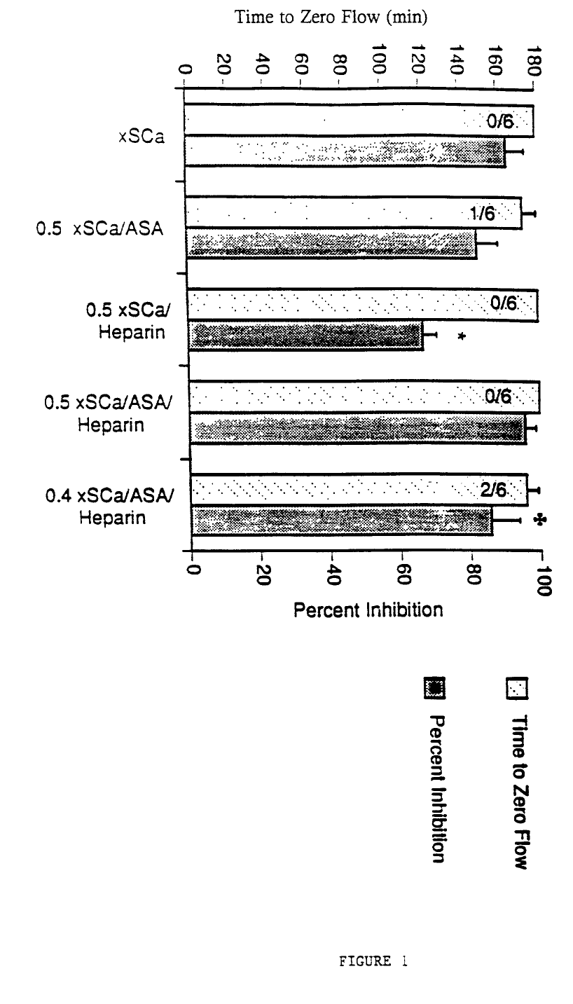

FIG. 1 compares the antithrombotic efficacy and the percent inhibition of platelet aggregation produced by SCa to that obtained by treatment with a decreased dose of SCa (0.5×SCa) combined with ASA, or heparin; or combined with ASA and heparin. The combination of 0.53SCa with ASA resulted in an occlusive thrombus in only 1 of the 6 dogs. When 0.5×SCa was administered with heparin, there was a significant reduction in the percent of steady-state inhibition of platelet aggregation relative 1×SCa (67±4% vs 92±5%) but the antithrombotic efficacy (100%) was similar to that of 1×SCa. The 0.5×SCa treatment combined with ASA and heparin was as effective as 1×SCa in preventing LCCA thrombosis. A further decrease from 0.5×SCa to 0.4×SCa with the ASA and heparin combination was less efficacious as there was LCCA occlusion in 2 of the 6 dogs. The maximum steady-state inhibition of platelet aggregation (96±3%) was observed in the group of dogs treated with 0.5×SCa combined with ASA and heparin.

Cyclic Flow Variations During Stimulation of the LCCA

Table 3 summarizes the CFV observed during anodal stimulation of the LCCA. As indicated in Table 3, CFV were observed in only 1 of 6 dogs from the groups treated with 1×SCa, 0.5×SCa combined with ASA, or 0.5×SCa in combination with ASA and heparin. The number of CFV was also significantly smaller in these groups compared to that observed in the groups treated with either ASA, heparin or ASA combined with heparin.

TABLE 3
Mean Cyclic Flow Variations Observed During Anodal
Stimulation of the LCCA in Anesthetized Dogs

| Intravenous | Mean CFV/Minute | Number of Dogs |
|---|---|---|
| treatment | (×100) | with CFV |
| 1 × SCa | 0.7 ± 0.7*†‡ | 1/6 |
| 0.6 × SCa | 3 ± 1 | 4/6 |
| 0.5 × SCa | 5 ± 1 | 6/6 |
| ASA | 6 ± 2 | 6/6 |
| Heparin^{a} | 5 ± 1 | 6/6 |
| ASA + Heparin | 7 ± 2 | 6/6 |
| 0.5 × SCa + ASA | 0.3 ± 0.3*†‡ | 1/6 |
| 0.5 × SCa + Heparin | 1 ± 1 | 3/6 |
| 0.5 × SCa + ASA + | 0.3 ± 0.3*†‡ | 1/6 |
| Heparin | | |
| 0.4 × SCa + ASA + | 4 ± 2 | 4/6 |
| Heparin | | |
| Saline | 5 ± 2 | 5/6 |

^{a}= 200 units/kg bolus followed by 1000 units/hourly (bolus); LCCA = left circumflex coronary artery; CFV = cyclic flow variations. Doses are shown in Table 1. Values are given in mean ± SEM. (n = 6/group).
*p < 0.05 vs ASA.
†p < 0.05 vs Heparin.
‡p < 0.05 vs ASA + Heparin.

Plasma Levels of SCa

Table 4 shows the results of plasma levels of SCa with the corresponding inhibition of platelet aggregation at steady-state conditions. The dose-dependent increase in mean percent inhibition of platelet aggregation was associated with a dose-dependent elevation of plasma SCa levels.

TABLE 4
Plasma Levels of SCa with the Related Ex Vivo
Inhibition of Collagen-Induced Platelet Aggregation

| | Plasma Level | Percent |
|---|---|---|
| Group | (ng/ml) | Inhibition |
| 1 × SCa | 57 ± 4 | 92 ± 5 |
| 0.6 × SCa | 48 ± 7 | 83 ± 3 |
| 0.5 × SCa | 35 ± 8 | 70 ± 4 |

SCa = 3S-[[4-[[4-aminoiminomethyl)phenyl]amino]-1,4-dioxobutyl]amino]-4-pentynoic acid. Percent inhibition represents steady-state condition. Values are given in mean ± SEM.

Antithrombotic therapy with ASA, heparin, or the combination is only partially effective in the prevention of coronary thrombus formation. Development of more effective antithrombotic and anticoagulant agents or combinations of both agents is desired. Platelet binding of fgn, by means of the RGD recognition sequence of the GPIIb/IIIa-receptor complex represents the final pathway of platelet aggregation and subsequent thrombus formation. This final pathway is common to all known platelet agonists. Therefore, the binding of fgn to GPIIb/IIIa receptors provides an excellent target for therapeutic intervention in thrombosis-related disorders such as the acute ischemic coronary syndromes. Several molecules have been shown to block fgn binding to platelet GPIIb/IIIa receptors and therefore prevent the formation of platelet thrombi.

Treatment of anesthetized dogs with the three different regimens of SCa achieved a dose-dependent, steady-state inhibition of ex vivo platelet aggregation which resulted in a dose-related sustained antithrombotic effect. The dose of SCa leading to approximately 90% inhibition of platelet aggregation appears to be completely protective. A salient finding is that 0.5×SCa given together with low dose ASA, or with heparin, or with ASA and heparin prevented arterial occlusion in 17 of 18 dogs. Since ASA, heparin, or ASA combined with heparin were not effective in this model, and 4 of 6 dogs incurred LCCA thrombosis after 0.5×SCa, the data suggest an enhanced antithrombotic effect between SCa, ASA and heparin.

Although both treatments of 0.5×SCa combined with heparin and 0.5×SCa administered with ASA plus heparin maintained coronary artery patency, the latter regimen showed a reduction in dogs having CFV (3 of 6 vs 1 of 6 dogs, respectively). The reduction of 1×SCa to 0.4×SCa combined with ASA and heparin provides less efficacy and increased CFV relative to the 0.5×SCa combinations. These data suggest an enhanced antithrombotic effect of SCa, ASA and heparin used in combination.

The present data shows that 3S-[[4-[[4-aminoiminomethyl)phenyl]amino]-1,4-dioxobutyl]amino]-4-pentynoic acid, a GPIIb/IIIa receptor antagonist, yields sustained levels of inhibition of ex vivo platelet aggregation that result in antithrombotic efficacy in a canine model of coronary artery occlusion. Furthermore, because of the very different mechanisms of action of SCa, ASA or heparin, the 0.5×SCa dose combined with these agents provide a protective antithrombotic effect, suggesting that decreased doses of the drug may be used in conjunction with ASA and heparin in the clinic for acute thrombotic-related events, Neither heparin nor ASA alone is efficacious in this model.

What we claim is:

1. A method for inhibiting platelet aggregation which method comprises administering to a mammal in need of such treatment a therapeutically effective amount of a compound selected from the group consisting of 3S-[[4-[[4-aminoiminomethyl)phenyl]amino]-1,4-dioxobutyl]amino]-4-pentynoic acid and ethyl 3S-[[4-[[4-(aminoiminomethyl)phenyl]amino]-1,4-dioxobutyl]amino]-4-pentynoate or pharmaceutically acceptable salts thereof;
and a therapeutically effective amount of an anti-coagulent and an anti-platelet agent.

2. A method according to claim 1 wherein the anti-platelet agent is aspirin.

3. A method according to claim 1 wherein the anti-coagulent is heparin.

4. A method according to claim 3 wherein the anti-platelet agent is aspirin.

5. A method for inhibiting platelet aggregation which method comprises administering to a mammal in need of such treatment a therapeutically effective amount of a compound selected from the group consisting of 3S-[[4-[[4-aminoiminomethyl) phenyl] amino]-1,4-dioxobutyl]amino]-4-pentynoic acid and ethyl 3S-[[4-[[4-(aminoiminomethyl)phenyl]amino]-1,4-dioxobutyl] amino]-4-pentynoate or pharmaceutically acceptable salts thereof;
and a therapeutically effective amount of an anti-coagulant agent.

6. The method according to claim 5 wherein the anti-coagulant agent is heparin.

7. A method for inhibiting platelet aggregation which method comprises administering to a mammal in need of such treatment a therapeutically effective amount of a compound selected from the group consisting of 3S-[[4-[[4-aminoiminomethyl)phenyl]amino]-1,4-dioxobutyl]amino]-4-pentynoic acid and ethyl 3S-[[4-[[4-(aminoiminomethyl)phenyl]amino]-1,4-dioxobutyl]amino]-4-pentynoate or pharmaceutically acceptable salts thereof;
and a therapeutically effective amount of an anti-platelet agent.

8. The method according to claim 7 wherein the anti-platelet agent is aspirin.
